(19) United States
(12) Patent Application Publication
(10) Pub. No.: US 2005/0222182 A1
(43) Pub. Date: Oct. 6, 2005

(54) Stabilized pharmaceutical compositions of halofuginone and other quinazolinone derivatives

(76) Inventors: Shai Yarkoni, Kfar Saba (IL); Lior Zelikovich, Mazkeret Batya (IL)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/505,071

(22) PCT Filed:
(86) PCT No.: PCT/IL03/00131

(30) Foreign Application Priority Data
Feb. 21, 2002 (IL) 148292

(51) Int. Cl. A61K 31/517

(52) U.S. Cl. 514/266.22

(57) ABSTRACT

The present invention relates to acid stabilized compositions containing as an active ingredient a quinazolinone derivative, preferably halofuginone or a pharmaceutically acceptable salt of halofuginone. More particularly the present invention relates to use of an acid for improving the stability of a topical, parenteral or oral composition containing quinazolinone derivatives as an active ingredient, preferably halofuginone. Surprisingly, even dry solid tablet dosage forms showed improved stability by addition of an acidic component.

### FIELD OF THE INVENTION

The present invention relates to acid stabilized compositions containing as an active ingredient a quinazolinone derivative, preferably halofuginone or a pharmaceutically acceptable salt of halofuginone. More particularly the present invention relates to use of an acid for improving the stability of a topical, parenteral or oral composition containing quinazolinone derivatives as an active ingredient, preferably halofuginone.

### BACKGROUND OF THE INVENTION

Halofuginone was originally developed as an oral anti-parasitic drug in veterinary applications.

U.S. Pat. No. 3,320,124, disclosed and claimed a method for treating coccidiosis with quinazolinone derivatives. Halofuginone, otherwise known as 7-bromo-6-chloro-3-[3-(3-hydroxy-2-piperidinyl)-2-oxopropyl]-4(3H)-quinazolinone (one of the quinazolinone derivatives), was first described and claimed in said patent of American Cyanamid company, and was the preferred compound taught by said patent and the one commercialized from among the derivatives described and claimed therein. Subsequently, U.S. Pat. Nos. 4,824,847; 4,855,299; 4,861,758 and 5,215,993 all related to the coccidiocidal properties of halofuginone. It has been marketed for veterinary use under the commercial name Steronol^{R}, and is still being used at the present time as an additive in chicken feed. Consequently, a substantial body of knowledge exists regarding the chemical characterization, toxicology and pharmacokinetics of the compound (NADA document #130-951 (SBA), 1985). U.S. Pat. No. 4,340,596 further discloses the use of lactate salts of quinazolinone derivatives for the treatment of a cattle disease caused by different types of theileria.

More recently, it was disclosed in U.S. Pat. No. 5,449,678 that these quinazolinone derivatives are unexpectedly useful for the treatment of a fibrotic condition. The '678 disclosure provides compositions of a specific fibrosis inhibitor comprising a therapeutically effective amount of a pharmaceutically active compound of the general formula I: 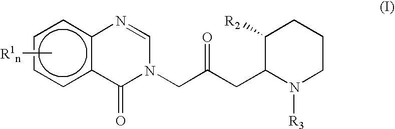
wherein: n=1-2
R₁ which may be the same or different at each occurrence is a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl.

Pharmaceutically acceptable salts thereof are also included. Of this group of compounds, halofuginone has been found to be particularly effective.

U.S. Pat. No. 5,449,678 discloses that these compounds are effective in the treatment of fibrotic conditions such as scleroderma and graft versus host disease (GVHD).

The ability of extremely low concentrations of halofuginone to inhibit specifically collagen type I gene expression enables broad therapeutic utility of halofuginone as a novel antifibrotic drug.

Progressive fibroproliferative diseases such as liver cirrhosis, pulmonary and kidney fibrosis, scleroderma and a variety of other serious diseases, exhibit excessive production of connective tissues, which results in the destruction of normal tissue architecture and function. The fibrotic reaction is thought to involve the stimulative response of tissue cells resulting in increased proliferation as well as extracellular matrix (ECM) deposition. Collagen was found to be a major ECM molecule synthesized in fibrotic lesions.

U.S. Pat. No. 5,891,879 further discloses that these compounds are effective in treating restenosis. The two former conditions are associated with excessive collagen deposition, which can be inhibited by halofuginone. Restenosis is characterized by smooth muscle cell proliferation and extracellular matrix accumulation within the lumen of affected blood vessels in response to a vascular injury [Choi et al., Arch. Surg., 130:257-261, 1995]. One hallmark of such smooth muscle cell proliferation is a phenotypic alteration, from the normal contractile phenotype to a synthetic one. Type I collagen has been shown to support such a phenotypic alteration, which can be blocked by halofuginone [Choi et al., Arch. Surg., 130:257-261, 1995; U.S. Pat. No. 5,449,678].

Notably, halofuginone inhibits collagen synthesis by fibroblasts in vitro, however, it promotes wound healing in vivo (WO 01/17531). Thus, the exact behavior of halofuginone in vivo cannot always be accurately predicted from in vitro studies.

In addition, quinazolinone containing pharmaceutical compositions including halofuginone have been disclosed and claimed as effective for treating malignancies (U.S. Pat. No. 6,028,075) as well as for prevention of neovascularization (U.S. Pat. No. 6,090,814).

The pathophysiological response to tissue trauma may differ in these tissues, but often results in the formation of scars or other types of connective tissues which lack the functionality of the original organ tissue, so that the repair of tissue trauma does not lead to a complete restoration of organ capacity and function.

In addition to the fibrotic diseases with excess collagen deposition, normal wound healing involves the formation of connective tissue that consist largely of collagen fibrils. Although moderate degrees of fibrous tissue are beneficial in wound repair, fibrous material often accumulates in excessive amount and impairs the normal function of the affected tissue. Such excessive accumulation of collagen becomes an important event in scarring of the skin after burns or traumatic injury, in hypertrophic scars and in keloids.

Currently available treatments to inhibit various abnormal responses to tissue trauma, such as the formation and growth of keloids and hypertrophic scars, cardiac fibrosis and other types of fibrotic disease processes, are generally unsatisfactory.

Other forms of treatment include the administration of corticosteroids. For example, triamcinolone appears to reduce the size of keloids and hypertrophic scars by increasing the rate of collagen degradation [Rockwell et al., Plastic and Recon. Surg., Vol. 84,827-835, 1989]. However, the side effects of such medications are potentially dangerous and treatments are not universally successful. Other treatments, such as radiation, also showed variable effectiveness and are associated with other potential side effects [Rockwell et al., Plastic and Recon. Surg., Vol. 84, 827-835, 1989]. Thus, clearly improved treatments for these diseases, which are associated with pathophysiological fibrotic processes, are required.

Although the pharmacological actions of halofuginone and its therapeutic effectiveness in various diseases were extensively studied, very little is known about the stability of this compound in pharmaceutical compositions, particularly its stability in the presence of an aqueous environment.

Halofuginone may be administered orally, parenterally or topically. These three dosage forms are distinct from one another in many ways. Formulations for oral administration should be easy to consume, non-irritant to the gastro-intestinal system, with the active ingredient resistant to metabolism and yet readily absorbed. Formulations for parenteral administration may be the most adequate for water-soluble drugs, however parenteral administration is less convenient and comfortable. Topical application may overcome few of the disadvantages of oral and parenteral applications, being comfortable and avoiding the gastro-intestinal system; however, molecules moving from the environment into and through intact skin must first penetrate the stratum corneum, which presents a barrier to absorption of topical compositions. In addition, formulations for topical application must take in consideration that the active ingredient is exposed to ambient temperature and light.

In pre-clinical trials mice were fed with halofuginone, or it was administered parenterally, without any formulation details. For topical application in preclinical studies and phase I clinical studies, the arbitrarily chosen vehicle was a cream of the type known as a water-in-oil (non-washable) cream, described in the USP as a “cold cream”, comprising beeswax, paraffin and borax in water. In a stability study, the active drug was found unstable in accelerated conditions at 40° C., as well at 25° C. and even at +4° C. No long-term stability studies of this formulation have been reported to date.

Since halofuginone has now been found to be effective in the treatment of diseases which are associated with pathophysiological fibrotic processes, as well as in restenosis and proliferative diseases, stable formulations of this compound are desired.

Thus, there is a recognized need for, and it would be highly advantageous to have suitable stable pharmaceutical compositions containing as an active ingredient quinazolinone derivatives, especially halofuginone, for topical application, parenteral or oral administration.

### SUMMARY OF THE INVENTION

The present invention deals with stabilized pharmaceutical compositions comprising quinazolinones having a piperidinic ring as substituent. The present invention discloses stabilized compositions and methods for decreasing the rate of isomerization of the trans isomer to the cis isomer by addition of an acidic component to the pharmaceutical composition and creating an acidic environment for the drug in different final dosage forms.

The present invention relates to pharmaceutical compositions comprising as an active ingredient a quinazolinone derivative having the general formula (I): 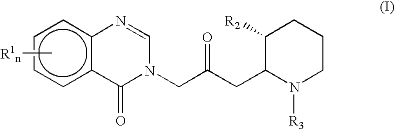
wherein: n=1-2
R₁ which may be the same or different at each occurrence is a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof, comprising a pharmaceutical acceptable carrier or diluent, further comprising an acid compound, providing a pH below 7.0.

Preferably the pH of the pharmaceutical composition will be below 6.5, more preferably below 6.0, and most preferably below 5.5.

The active compound is preferably halofuginone or a pharmaceutically acceptable salt of halofuginone.

More particularly the present invention relates to topical, parenteral and oral compositions containing halofuginone and an acidic compound, wherein the pH is below 7.0. Preferably the pH of the pharmaceutical composition will be below 6.5, more preferably below 6.0, and most preferably below 5.5.

The present invention provides a method for stabilizing a quinazolinone derivative having the general formula (I) 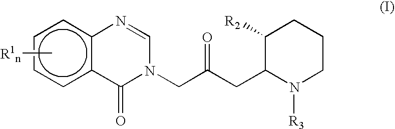
wherein: n=1-2
R₁ which may be the same or different at each occurrence is a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof, comprising formulating said quinazolinone in a pharmaceutical acceptable carrier or diluent, further comprising an acid compound, thereby providing a pH below 7.0.

Preferably the pH of the formulation will be below 6.5, more preferably below 6.0, most preferably below 5.5. The role of the acid compound, providing the acid pH is to improve the stability of the active compound for storage purposes and for enhanced drug performance.

The pH of the pharmaceutical compositions according to the methods of the present invention are preferably in the range of 2.5-6.5, more preferably in the range of 3.5 to 5.5. The role of the acid compound, providing the acid pH is to improve the stability of the active compound for storage purposes and for enhanced drug performance.

The active compound is preferably halofuginone, or a pharmaceutically acceptable salt of halofuginone.

The acid compound for use in the formulations of the invention is preferably selected from the group consisting aliphatic, aromatic, acetic, ascorbic, benzoic, citric, glycolic, lactic, malic, maleic, and stearic acid, though any pharmaceutically acceptable acid may be used.

In certain preferred embodiments the compound of general formula I is conveniently formulated for topical use. The pharmaceutical compositions of the present invention formulated for topical use are preferably selected from the group consisting of cream, ointment, lotion, gel, suspension, aqueous or cosolvent solutions, salve, liposomes and any other pharmaceutically acceptable carrier suitable for administration of the drug topically. More preferred topical formulations are selected from the group consisting of emulsions, non-washable (water-in-oil) creams or washable (oil-in-water) creams, lotions, salves, gels and the like. One currently more preferred embodiment of the invention provides a topical formulation comprising a gel having a pH below 5.5.

In additional and alternative preferred embodiments the compound of formula I is formulated for parenteral use. The pharmaceutical compositions for parenteral administration are preferably selected from the group consisting of sterile solutions ready for injection, sterile suspensions ready for injection, sterile dry soluble lyophilized powders ready for reconstitution by combination with a vehicle just prior to use, sterile emulsions, microemulsions, dispersions, liposomal delivery systems, lipid complexes such as with cholesterol derivatives and phospholipids.

Pharmaceutical compositions for parenteral administration are formulated in forms suitable for intravenous injections, intravenous infusion, intradermal, intralesional, intramuscular, and subcutaneous injections or depots; or they may be administered parenterally by means other than an injection, for example, laparascopically, intravesicularly, or via any orifice not related to the gastrointestinal tract.

In additional and alternative preferred embodiments the compound of formula I is formulated for oral administration. Pharmaceutical compositions for oral administration are formulated in aqueous or solid forms. Aqueous dosage forms suitable for oral administration are in all essential ingredients identical to those of parenteral aqueous dosage forms, with the exception that flavoring agents or coloring agents may be optionally added.

Currently more preferred embodiments of the present invention relate to a solid pharmaceutical composition selected from the group consisting of tablets, capsules, sachets, powders, granules and lozenges.

In dry solid pharmaceutical formulations the acid component will provide the necessary acid environment only in the presence of moisture. Unexpectedly, it is now disclosed that even in such dry solid formulations the presence of an acidic component is essential to ensure long-term stability, providing a shelf life compatible with the commercial development of the product.

### BRIEF DESCRIPTION OF THE FIGURES

The invention is herein described by way of example only, with reference to the accompanying drawings, wherein:

FIG. 1 Shows the effect of solution pH and ambient temperature on halofuginone stability.

FIG. 2 Demonstrates the higher stability of halofuginone in lactate buffer pH 4.0 compared to distilled water.

FIG. 3 Shows that halofuginone stability is not affected by the addition of saline to lactate buffer.

### DETAILED DESCRIPTION OF THE INVENTION

The present invention relates to pharmaceutical compositions comprising as an active ingredient a quinazolinone derivative having the general formula (I): 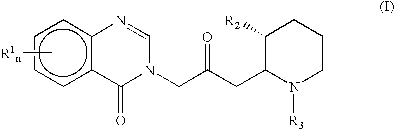
wherein: n=1-2
R₁ which may be the same or different at each occurrence is a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof, comprising a pharmaceutical acceptable carrier or diluent, further comprising an acid compound, providing a pH below 7.0.

Preferably the pH of the pharmaceutical composition will be below 6.5, more preferably below 6.0, most preferably below 5.5. The role of the acid compound, providing the acid pH is to improve the stability of the active compound for storage purposes and for enhanced drug performance.

The active compound is preferably halofuginone, or a pharmaceutically acceptable salt of halofuginone.

More particularly the present invention relates to pharmaceutical compositions for topical, parenteral or oral administration containing a quinazolinone derivative, preferably halofuginone, an acid compound and pharmaceutically acceptable carriers.

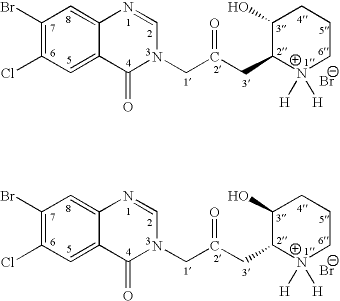

As described in the scheme above, halofuginone is an alkaloid containing a quinazolin-4-one moiety that is connected to a piperidinic ring by a ketonic bridge. The piperidinic ring is substituted on position 3″ with a hydroxyl group.

There are two chiral atoms in the molecule at positions 2″ and 3″ of the piperidinic ring, whereas halofuginone is synthesized as racemate at position 3″. The configuration at carbon 2″ interconverts spontaneously so that the configuration of the substituents interconvert between cis and trans as described in the following scheme. 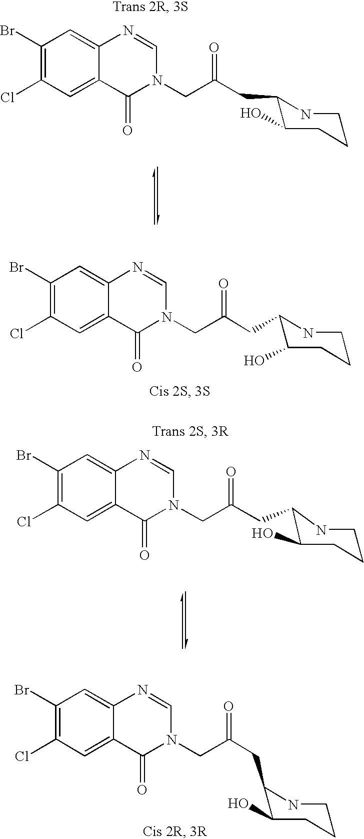
The mechanism of isomerization is described in the scheme below:

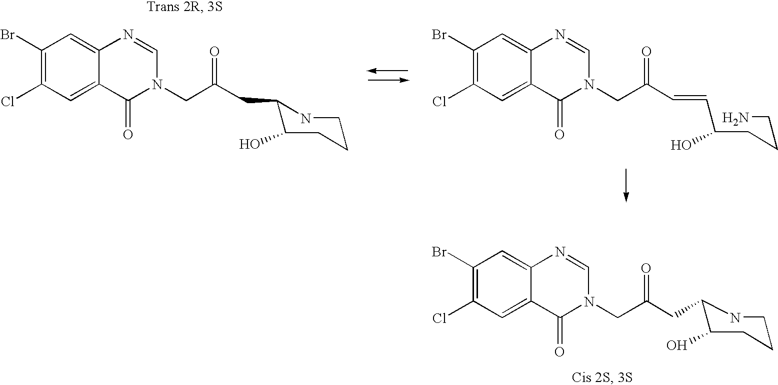

Protonation of the piperidinic nitrogen atom prevents the ring opening and therefore the isomerization rate is decreased. The rate of the equilibration process between the two diasteriomers dictates the ratio of the isomers that will be formed due to this process rate.

The present invention deals with quinazolinones having a piperidinic ring as substituent. In this invention we disclose compositions and methods for decreasing the rate of isomerization of the pharmaceutically active trans isomer to the cis isomer by addition of an acidic component to the pharmaceutical composition and creating an acidic environment for the drug. The present invention further discloses that the isomerization rate of salts of quinazolinones towards the cis isomer is also reduced under acidic conditions.

The present invention provides a method for stabilizing a quinazolinone derivative having the general formula (I): 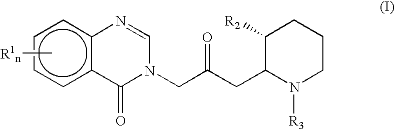
wherein: n=1-2
R₁ which may be the same or different at each occurrence is a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof, in a pharmaceutical acceptable composition, by adding an acid compound, providing a pH below 7.0.

More preferably the pH of the pharmaceutical composition will be below 6.5, most preferably below 6.0.

The role of the acid compound, providing the acid pH is to improve the stability of the active compound for storage purposes and for enhanced drug performance.

The active compound is preferably halofuginone, or a pharmaceutically acceptable salt of halofuginone.

In preferred embodiment the present invention provides a pharmaceutical composition for topical, parenteral or oral administration.

In preferred embodiments according to the present invention, halofuginone is chemically stable in the compositions, when the pH of the composition is acidic, the pH of the composition is in the range of pH 1 to pH 6.8, preferably the pH is in the range of pH 3.5 to 6.0. “Improved stability” is used herein to denote that the amount of the active ingredient remaining in the pharmaceutical active form is significantly higher over a given period of time compared to a control without the acidic component.

In preferred embodiments according to the present invention the halofuginone concentration in the composition is in the range 0.0001-30% (w/w).

According to a more preferred embodiment the halofuginone concentration in the composition is in the range of 0.001 to 10%.

Topical Application

Preferred embodiments of the present invention relate to topical pharmaceutical compositions comprising as an active ingredient a quinazolinone derivative having the formula: 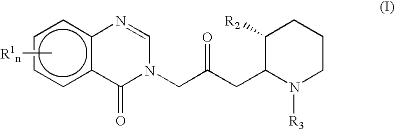
wherein: n=1-2
R₁ which may be the same or different at each occurrence is a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof, in a pharmaceutical acceptable carrier or diluent, further comprising an acid compound, providing a pH below 7.0.

The active compound is preferably halofuginone, or a pharmaceutically acceptable salt of halofuginone.

The pharmaceutical compositions of the present invention formulated for topical use are selected from the group consisting of cream, ointment, lotion, gel, suspension, aqueous or cosolvent solutions, salve, liposomes and any other pharmaceutically acceptable carrier suitable for administration the drug topically.

In more preferred embodiments the topical formulation is selected from the group consisting of emulsions, non-washable (water-in-oil) creams or washable (oil-in-water) creams, lotions, salves and the like.

As is well known in the art the physico-chemical characteristics of the carrier may be manipulated by addition a variety of excipients, including but not limited to thickeners, gelling agents, wetting agents, flocculating agents, suspending agents and the like. These optional excipients will determine the physical characteristics of the resultant formulations such that the application may be more pleasant or convenient. It will be recognized by the skilled artisan that the excipients selected, should preferably enhance and in any case must not interfere with the storage stability of the formulations.

The halofuginone concentration in the topical compositions is in the range 0.0001-10% (w/w) and most preferably in the range of 0.001-2% (w/w).

The acid compound for use in the topical formulations of the invention is selected from the group consisting aliphatic acids, aromatic, acetic, ascorbic or benzoic acid, citric, glycolic, lactic, malic and maleic acid.

According to one currently preferred embodiment, the acid compound for use in the topical formulation of the invention is lactic acid.

A first preferred embodiment according to the present invention is a cream formulation comprising in addition to the active compound: (a) a hydrophobic component; (b) a hydrophilic aqueous component; and (c) at least one emulsifying agent, wherein the pH of the aqueous component is less than 7.0.

Preferably the hydrophobic component of the cream is present in an amount from about 10% to about 90% (w/w) based on the total weight of the composition and most preferably the hydrophobic component of the cream is present in an amount from about 20% to about 80% (w/w) based on the total weight of the composition.

The hydrophobic component of the cream is exemplified by the group consisting of mineral oil, yellow soft paraffin (Vaseline), white soft paraffin (Vaseline), paraffin (hard paraffin), paraffin oil heavy, hydrous wool fat (hydrous lanolin), wool fat (lanolin), wool alcohol (lanolin alcohol), petrolatum and lanolin alcohols, beeswax, cetyl alcohol, almond oil, arachis oil, castor oil, hydrogenated castor oil wax, cottonseed oil, ethyl oleate, olive oil, sesame oil, and mixtures thereof.

The hydrophilic aqueous component of the cream is exemplified by water alone, propylene glycol or alternatively any pharmaceutically acceptable buffer or solution.

Exemplary buffers are acetate, borate (borax), citrate, lactate, phosphate and mixtures thereof.

In a preferred embodiment the hydrophilic aqueous component of the cream is present in an amount from about 10% to about 90% (w/w) based on the total weight of the composition and most preferably the hydrophilic aqueous component of the cream is present in an amount from about 20% to about 80% (w/w) based on the total weight of the composition.

Emulsifying agents are added to the cream in order to stabilize the cream and to prevent the coalescence of the droplets. The emulsifying agent reduces the surface tension and forms a stable, coherent interfacial film.

The cream composition contains at least one emulsifying agent, preferably the emulsifying agent is a complex emulgator which comprises a combination of a hydrophilic and a hydrophobic emulsifying agent.

In a preferred embodiment the complex emulgator is present in an amount effective to stabilize the cream formed from the hydrophobic component and hydrophilic aqueous component.

In a preferred embodiment the ratio of the hydrophilic and hydrophobic emulsifying agents comprising the complex emulgator depends on the type of cream formulated (i.e. oil-in-water and water-in-oil) and on the required HLB (hydrophilic-lipophilic balance) of the inner (discontinuous) emulsified phase. In a preferred embodiment the concentration of the complex emulgator in the cream is in the range from about 2% to about 40% (w/w) based on the total weight of the composition.

Suitable hydrophilic emulsifying agents comprising the complex emulgator may be exemplified but not limited to the group consisting of polyoxyethylene sorbitan monolaurate (Tween 20), polyoxyethylene sorbitan monopalmitate (Tween 40), polyoxyethylene sorbitan monostearate (Tween 60), polyoxyethylene sorbitan monooleate (Tween 80), polyoxyethylene lauryl ether (Brij 35), polyoxyethylene castor oil (Atlas G-1794), sodium lauryl sulfate, cetrimide, cetomacrogol and mixtures thereof.

Suitable hydrophobic emulsifying agents comprising the complex emulgator may be exemplified but not limited to the group consisting of sorbitan trioleate (Span 85, Aracel 85), sorbitan tristearate, sorbitan sesquioleate (Aracel 83), (Span 65), sorbitan monooleate (Span 80), propylene glycol monostearate, sorbitan sequioleate (Aracel C), glycerol monostearate, propylene glycol monolaurate (Atlas G-917, Atlas G-3851), sorbitan monostearate (Span 60, Aracel 60), sorbitan monopalmitate (Span 40, Aracel 40), sorbitan monolaurate (Span 20, Aracel 20), cetostearyl alcohol, cetyl alcohol, oleic acid, stearic acid and mixture thereof.

A suitable emulsifying agent may be exemplified by but not limited to the group consisting of cholesterol, cetostearyl alcohol, wool fat (lanolin), wool alcohol (lanolin alcohol), hydrous wool fat (hydrous lanolin), and mixtures thereof.

In a preferred embodiment the concentration of the at least one emulsifying agent in the cream is in the range from about 2% to about 40% (w/w) based on the total weight of the composition.

In another preferred embodiment pharmaceutical composition of the present invention is formulated in the form of aqueous suspensions.

In a preferred embodiment the suspension comprising in addition to the active compound: (a) an aqueous medium; and (b) suspending agents or thickeners. (c) an acid compound, optionally additional excipients are added, as specified heretofore.

Suitable suspending agent or thickeners may be exemplified by but not limited to the group consisting of cellulose derivatives like methylcellulose, hydroxyethylcellulose and hydroxypropyl cellulose, alginic acid and its derivatives, xanthan gum, guar gum, gum arabic, tragacanth, gelatin, acacia, bentonite, starch, microcrystalline cellulose, povidone and mixture thereof.

In preferred embodiments the suspending agents or thickeners are present in an amount from about 0.1% to about 15% (w/w) based on the total weight of the composition.

In preferred embodiment the aqueous suspensions may optionally contain additional excipients selected from the group consisting of wetting agents, flocculating agents, thickeners, and the like.

Suitable wetting agents are exemplified by but not limited to the group consisting of glycerol polyethylene glycol, polypropylene glycol and mixtures thereof, and surfactants. The concentration of the wetting agents in the suspension should be selected to achieve optimum dispersion of the pharmaceutical powders within the suspension with the lowest feasible concentration of the wetting agent.

Suitable flocculating agents are exemplified by but not limited to the group consisting of electrolytes, surfactants, and polymers.

The suspending agents, wetting agents and flocculating agents are provided in amounts that are effective to form a stable suspension of the pharmaceutically effective agent.

In another preferred embodiment the gel formulation of the present invention comprising in addition to the active compound, at least one gelling agent and an acid compound.

Suitable gelling agents may be exemplified by but not limited to the group consisting of hydrophilic polymers, natural and synthetic gums, crosslinked proteins and mixture thereof. In a preferred embodiment the polymers are selected from the group consisting of hydroxyethylcellulose, hydroxyethyl methylcellulose, methyl cellulose, hydroxypropylcellulose, hydroxypropyl methylcellulose, carboxymethyl cellulose, and similar derivatives of amylose, dextran, chitosan, pullulan, and other polysaccharides; Crosslinked proteins such as albumin, gelatin and collagen; acrylic based polymer gels such as Carbopol, Eudragit and hydroxyethyl methacrylate based gel polymers, polyurethane based gels and mixtures thereof.

In a preferred embodiment the gums are selected from the group consisting of acacia, agar, carageenan, dextrin, gelatin, guar gum, hyaluronic acid, tragacanth gum, xanthan gum, and mixtures thereof. In a preferred embodiment the gelling agent is present in an amount from about 1% to about 25% (w/w) based on the total weight of the composition. In a preferred embodiment the pH of the aqueous phase of the gel is in the range 1.0-6.8.

In another preferred embodiment pharmaceutical compositions of the present invention are formulated as a solution. Such a solution comprises, in addition to the active compound, an acid compound and at least one co-solvent exemplified but not limited to the group consisting of water, buffered solutions, organic solvents such as ethyl alcohol, isopropyl alcohol, propylene glycol, polyethylene glycol, glycerin, glycoforol, Cremophor, ethyl lactate, methyl lactate, N-methylpyrrolidone, ethoxylated tocopherol and mixtures thereof.

In a preferred embodiment the solution comprises a mixture of the active agent dissolved or dispersed in an aqueous solution of a pH range between 1 and 6.8. Alternatively cosolvent solutions of halofuginone may be prepared using pharmaceutically acceptable organic solvents such as ethanol, isopropanol, glycerol, propylene glycol, low molecular weight poly(ethylene glycol) and its copolymers with propylene glycol. The solutions may be a maintained as a mixture of hydrophilic components or contain water at various amounts for oral and systemic as well as topical use.

The preferred concentration of halofuginone in solutions is between 0.0001 to about 1 weight %. The solutions containing water should be kept at a pH range between 1 and 6.8 to avoid irritation or degradation of the drug, halofuginone.

The topical composition of the present invention may optionally contain at least one additional ingredient, selected from the group consisting of advantageously preservatives, antioxidants, humectants, emollients, thickeners, structuring agents, stabilizers, coloring agents, and perfumes.

The creams, ointments, lotions and gels may be prepared by incorporating halofuginone in a finely-divided or powdered form alone or in solution or suspension, in an aqueous or non-aqueous fluid, to the pharmaceutical carrier.

Halofuginone may be dissolved, dispersed, suspended or partially dispersed and partially dissolved in the pharmaceutical carrier, depending on the solubility of halofuginone in the selected pharmaceutical carrier.

It is understood that the precise dosage and duration of treatment is a function of the tissue being treated. It is to be noted that concentrations and dosage values may also vary with the age, weight and condition of the individual treated. It is to be further understood that for any particular subject, specific dosage regimens should be adjusted over time according to the individual need and professional judgment of the physician or person administering or supervising the administration of the formulations.

Parenteral Administration

Alternative preferred embodiments of the present invention relate to pharmaceutical compositions for parenteral administration, comprising as an active ingredient a quinazolinone derivative having the general formula (I): 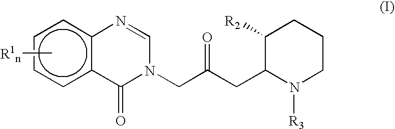
wherein: n=1-2
R₁ which may be the same or different at each occurrence is a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof, in a pharmaceutically acceptable carrier further comprising an acid compound providing a pH below 7.0.

Preferably the pH is below 6.5 more preferably below 6.0. The role of the acid compound, providing the acid pH is to improve the stability of the active compound for storage purposes and for enhanced drug performance.

The active compound is preferably halofuginone, or a pharmaceutically acceptable salt of halofuginone.

Pharmaceutical compositions for parenteral administration are formulated for intravenous injections, intravenous infusion, intradermal, intralesional, intramuscular, and subcutaneous injections or depots; or they may be administered parenterally by means other than an injection, for example, it could be introduced laparascopically, intravesicularly, or via any orifice not related to the gastrointestinal tract.

In a preferred embodiment the pharmaceutical compositions for parenteral administration are preferably a formulation selected from the group consisting of sterile solutions ready for injection, sterile suspensions ready for injection, sterile dry soluble lyophilized powders ready for reconstitution by combination with a vehicle just prior to use, sterile emulsions, microemulsions, dispersions, liposomal dosage forms, lipid complexes such as with cholesterol derivatives and phospholipids.

In a preferred embodiment the solutions and vehicles are selected from the group consisting of aqueous or non-aqueous solutions. In a preferred embodiment the aqueous parenteral solutions and vehicles are selected from the group consisting of sterile water for injection, sodium chloride injection, Ringers injection, Isotonic Dextrose injection, Dextrose and Lactated Ringers injection.

In a preferred embodiment the aqueous parenteral solutions or vehicles further comprise buffering agents. In a more preferred embodiment, the pH of the aqueous parenteral solution is in the range of 3.5 to 6.0.

The acid compound, for the parenteral formulations, is selected from the group consisting of ascorbic and benzoic acid, citric glycolic, lactic, malic and maleic acid.

In a preferred embodiment, the aqueous parenteral vehicle may further comprise cosolvents also referred to as water miscible solvents such as ethyl alcohol, polyethylene glycol, propylene glycol and mixture thereof.

In a preferred embodiment the sterile injection may comprise lyophilized powders ready for reconstitution by aqueous vehicle. Such lyophilized powders comprising quinazolinone derivative and a solid pharmaceutically acceptable buffering agent or a water-soluble organic acid. The buffering agents or organic acids used in the composition may be any non-toxic buffering agent or organic acid approved for parenteral use.

In a preferred embodiment the buffering agent or organic acid are present in amount such that the pH of the formulation upon reconstitution with water or other pharmaceutically acceptable vehicle is between about 3.5 to about 6.0.

Optionally, at least one additional ingredient selected from the group consisting of preservatives, antioxidants and tonicity controlling agents can be used.

In a preferred embodiment the preservatives are selected from the group consisting of benzyl alcohol, methyl paraben, propyl paraben, sodium salts of methyl paraben.

In a preferred embodiment the tonicity controlling agents are selected from the group consisting of sodium chloride, mannitol, dextrose, glucose, lactose and sucrose.

Oral Administration

In additional and alternative preferred embodiments the present invention relates to pharmaceutical compositions for oral administration, comprising as an active ingredient a quinazolinone derivative having the general formula (I): 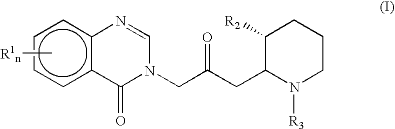
wherein: n=1-2
R₁ which may be the same or different at each occurrence is a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof, in a pharmaceutical acceptable carrier further comprising an acid compound, providing a pH lower than pH 7.0.

Preferably, the pH is below 6.5 more preferably below 6.0. The role of the acid compound, providing the acid pH, is to improve the stability of the active compound for storage purposes and for enhanced drug performance.

The active compound is preferably halofuginone, or a pharmaceutically acceptable salt of halofuginone.

Pharmaceutical compositions for oral administration are formulated as aqueous or solid dosage form.

Aqueous Dosage Form

In a preferred embodiment the pharmaceutical compositions for oral administration are formulated in a form selected from the group consisting of sterile solutions, sterile suspensions, sterile dry soluble lyophilized powders ready for reconstitution by combination with a vehicle just prior to use, sterile emulsions, microemulsions, dispersions, liposomal dosage forms, lipid complexes such as with cholesterol derivatives and phospholipids.

In a preferred embodiment the solutions and vehicles are selected from the group consisting of aqueous or non-aqueous solutions.

In a preferred embodiment the aqueous vehicle for oral solutions are selected from the group consisting of sterile water and sodium chloride.

In a preferred embodiment the aqueous oral solutions or vehicles further comprise buffering agents. In a more preferred embodiment, the pH of the aqueous oral solution is in the range of 3.5 to 6.0.

The acid compound, for the oral formulations, is selected from the group consisting of glycolic, lactic, malic, maleic, citric, ascorbic and benzoic acid.

In a preferred embodiment, the aqueous oral vehicle may further comprise cosolvents such as ethyl alcohol, polyethylene glycol, propylene glycol and mixture thereof.

In a preferred embodiment the sterile formulation may comprise lyophilized powders ready for reconstitution by aqueous vehicle. Such lyophilized powders comprising quinazolinone derivative and a solid pharmaceutically acceptable buffering agent or a water-soluble organic acid. The buffering agents or organic acids used in the composition may be any non-toxic buffering agent or organic acid approved for oral use.

In a preferred embodiment the buffering agent or organic acid are present in amount such that the pH of the formulation upon reconstitution with water or other pharmaceutically acceptable vehicle is between about 3.5 to about 6.0.

Optionally, at least one additional ingredient selected from the group consisting of preservatives, antioxidants and tonicity controlling agents.

In a preferred embodiment the preservatives are selected from the group consisting of benzyl alcohol, methyl paraben, propyl paraben, sodium salts of methyl paraben.

In a preferred embodiment the tonicity controlling agents are selected from the group comprising of sodium chloride, mannitol, dextrose, glucose, lactose and sucrose.

Solid Dosage Form

In preferred embodiment the present invention relates to a solid pharmaceutical composition selected from the group consisting of tablets, capsules, sachets, powders, granules and lozenges.

Tablets

In preferred embodiment the present invention relates to a solid pharmaceutical formulated as tablets containing in addition to the active compound suitable excipients including, but not limited to, starches, gum arabic, calcium silicate, microcrystalline cellulose, polyvinylpyrrolidone, cellulose, water, syrup, and methyl cellulose. The formulations can additionally include lubricating agents such as, for example, talc, magnesium stearate and mineral oil; wetting agents; emulsifying and suspending agents; preserving agents such as methyl- and propyl hydroxybenzoates; sweetening agents; or flavoring agents. Polyols, buffers, and inert fillers may also be used. Examples of polyols include, but are not limited to: mannitol, sorbitol, xylitol, sucrose, maltose, glucose, lactose, dextrose, and the like. Suitable buffers encompass, but are not limited to, phosphate, citrate, tartarate, succinate, and the like. Other inert fillers which may be used encompass those which are known in the art and are useful in the manufacture of various dosage forms. If desired, the solid pharmaceutical compositions may include other components such as bulking agents and/or granulating agents, and the like. The compositions of the invention can be formulated so as to provide quick, sustained, or delayed release of the active ingredient after administration to the patient by employing procedures well known in the art.

The acid compound is selected from the group consisting of ascorbic, citric maleic, and stearic, acid.

It is to be understood that the invention is not limited in its application to the details of construction and arrangement of the components set forth in the following description. The invention includes other embodiments and can be practiced or implemented in various ways. Also it is to be understood that the phraseology and terminology employed herein is for the purpose of description only and should not be regarded as limiting.

### EXAMPLES

Solutions

In solution the stability of the active ingredient of formula I is now shown to depend strongly on the pH as well as on previously known factors such as temperature and light. The protonation of the nitrogen under acidic condition protects the compound from isomerization. In basic conditions the salt is converted to the free base, which is highly sensitive to piperidine ring opening, following by isomerization to the cis-isomer. The stability of halofuginone-HBr salt has been studied in several formulations including solutions for injection, eye drops, creams and tablets.

Stability of Halofuginone-HBr in Aqueous Buffered Solutions

Purpose: To determine the hydrolytic stability of halofuginone in buffer solutions. These data are useful to determine stable topical and injectable formulations for the drug.

Solubility of Halofuainone-HBr in Various Solvents

The solubility of halofuginone in several solvents was first studied. Powder of halofuginone was dissolved in the different solvents, starting from over saturation and followed by dilutions.

Among the various materials tested, water was found to be the best solvent; mixture of water and other solvents showed synergistic effect with respect to the solubility in each individual solvent, as summarized in table 1.

According to this study, halofuginone solutions prepared by dissolving halofuginone in water buffered to pH 4 with lactic acid were employed for the preparation of the different dosage forms, as such solutions were found to be the most stable and effective.

TABLE 1
Solubility of halofuginone-Hbr

| | | Maximum | | |
|---|---|---|---|---|
| | | solubility | | |
| | | determined | Experiment Conditions | |
| No. | Solvent | (% w/v) | +50° C. | RT |
| 1 | Water buffered with lactic | 0.5 | O.K. | O.K. |
| | acid to pH = 3.5-4.0 | | | |
| | (Designated as W4) | | | |
| 2 | Ethanol absolute - Ethyl | 0.1 | Dissolved | Dissolved |
| | alcohol | | partially | partially |
| 3 | Isopropyl alcohol - 2- | 0.1 | Dissolved | Dissolved |
| | Propanol (IPA) | | partially | partially |
| 4 | Propylene glycol 1,2 | 0.25 | O.K. | O.K. |
| | Propane diol (PG) | | | |
| 5 | Polyethylene glycol 400 | 0.1 | O.K. | O.K. |
| | (PEG 400) | | | |
| 6 | Mix 1:1 vol W4:PEG 400 | 1.0 | O.K. | O.K. |
| 7 | Mix 1:1 vol W4:PG | 1.0 | O.K. | O.K. |
| 8 | Mix 1:1 vol W4:IPA | 1.0 | O.K. | O.K. |

Measurements of Halofuginone in Other Solutions

Solutions for stability measurements were prepared as follows:

Halofuginone HBr powder was dissolved in acetonitrile and the solution was mixed with 4 volumes (1:1 acetonitrile: buffer) of borate buffer to yield a 1 g/ml solution. Buffer solutions at pH 5.5, 6.5, and 7.5 were prepared by mixing various amounts of sodium borate solution (12 g in 240 ml DDW) to boric acid solution (12 g in 240 ml DDW). The solutions were filtered through a 0.2 micron filter into a tightly closed glass vial coated with aluminum foil to protect from light. The solutions (1.25 ml, 1 mg/ml in acetonitrile: buffer 1:4) were kept in temperature controlled incubators at 4, 25, 37 and 60° C. At time of analysis, about 50 μl of the solution was taken and immediately injected to the HPLC.

HPLC Analysis:

Halofuginone was determined by HPLC using the following equipment and conditions:
Instrument: HP 1050 with HPCHEM software
Column: C8, Lichospher 60 (5, μm) LichoCART 125-4, Cat. No. 1.50213 (Merck) (similar to Hypersil BDS C8 used by AminoLab) with precolumn Lichospher 100 RP-8, 5 μm, 4×4 mm, Cat. No. 1.50961 (Merck)
Column temp: ambient
Flow rate: isocratic, 1 ml/min Eluant: Acetonitrile: 0.1 M sodium citrate buffer pH3.0 at a 30:70 v/v
Detector: UV 254 nm
Sample volume: 20 μl
Run time: 15 min., halofuginone Rt=8.8 min.; degradation product Rt=11.7
   Results:

Analysis: Halofuginone was analyzed by HPLC with UV detector. The retention times for halofuginone and its main degradation product (cis isomer) are 8.8 min and 11.7 min, respectively.

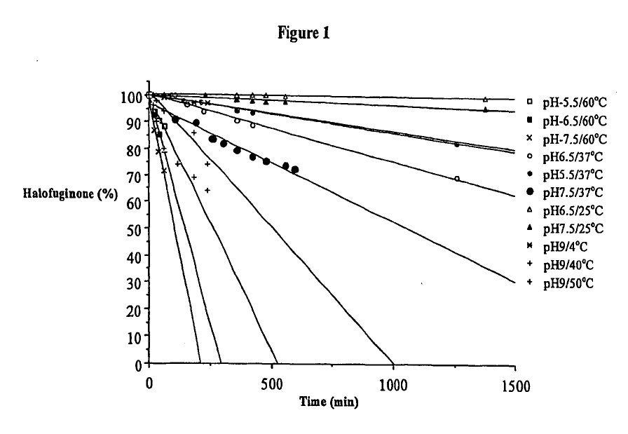

Stability: Halofuginone concentrations were followed for 24 hours. No change in halofuginone concentrations and no degradation products were found for the solutions kept at 4° C. and at pH 5.5 at 25° C. The decrease in halofuginone concentrations for the solutions kept at 25° C. and above is shown in FIG. 1 and the rate constants are summarized in Table 1. The degradation activation energies at different pH solutions were estimated from the plot of ln(K_{obs}) vs. 1/T and are summarized in Table 1. The activation energies were calculated from the slopes of ln(K_{obs}) vs. 1/T. The activation energies are in the range of 6.57-13.13 Kcal.

TABLE 2
Degradation rate (%/min) of halofuginone
at various pH and temperatures(FIG. 1)

| Temp | | | | |
|---|---|---|---|---|
| (° K) | 1/° K | pH 5.5 | pH 6.5 | pH 7.5 |
| 277 | | — | — | — |
| 289 | 0.0033557 | 0.00000 | 0.00077 | 0.00366 |
| 310 | 0.0032258 | 0.01415 | 0.02500 | 0.04435 |
| 333 | 0.0030030 | 0.18780 | 0.34335 | 0.46840 |
| Slope [ln(K_{obs})/1/T] | | 26,134 | 16,685 | 13,412 |
| Ea (K_{cal}) | | 13.13 | 8.38 | 6.57 |

These results show that halofuginone is stabilized at lower pH and its degradation is accelerated at even slightly basic conditions.

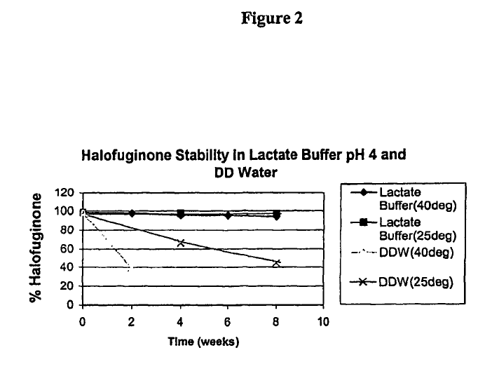

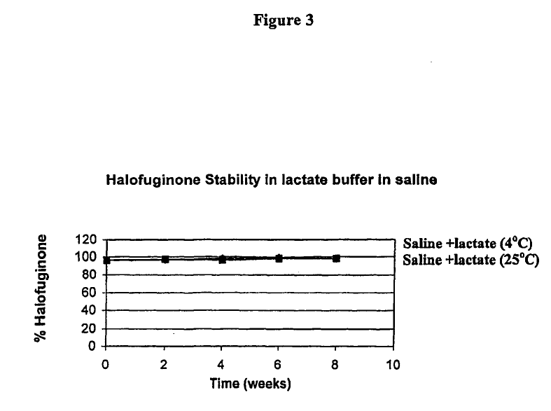

A commercially useful solution formulation was developed based on halofuginone dissolved in lactate buffer and the stability data shown in FIGS. 2 and 3. The stability of halofuginone in sodium lactate buffer (4% pH 4) was unaffected by the presence of saline in the final solution.

Preparation of Halofuginone Solution for Gavage

400 μl lactic acid was dissolved in 5 ml of double distilled (DD) water. The pH of the mixture was adjusted to pH 4.0-4.5 with ˜5001 NaOH 30% (w/v in DD water). The volume of the solution was brought to 10 ml with DD water. Halofuginone/HBr was added to a final concentration of 1 mg/ml (5 mg halofuginone in 5 ml of the above described solution). The solution was then heated at 40-45° C., for less than 1 minute until full dissolution. The resulting stock solution may be diluted with DD water to any required concentration before injection.

Preparation of Halofuginone Solution for Injection

400 μl lactic acid was dissolved in 5 ml of double distilled (DD) water. The pH of the mixture was adjusted to pH 4.0-4.5 with ˜500 μl NaOH 30% (w/v in DD water). The total volume of the solution was brought to 10 ml with DD water. Halofuginone/HBr was added to a final concentration of 1 mg/ml (5 mg halofuginone in 5 ml of the above described solution). The solution was then heated at 40-45° C., for less than 1 minute until full dissolution. The resulting stock solution was diluted with saline to any required concentration below 0.25 mg/ml and then filter-sterilized through 0.2μ filter.

Topical Application

Formulations for topical application may include gels, suspensions, aqueous or cosolvent solutions, salves, liposomes and creams.

Preliminary formulations for topical application of halofuginone were prepared in two directions: hydrophilic gel and lipophilic cream.

Hydrophilic gel—The gel was prepared with a jellifying substance: polyethylenglycol (Poloxamer-Lutrol F 127) or a synthetic cellulose derivative—Hydroxyethylcellulose—(Natrosol 250 HX Pharm). The first gel with Poloxamer—was found to be too sticky and contained a large amount of entrapped air. The gel prepared with Natrosol was found to be more suitable for topical application.

The ingredients of this vehicle, meaning water, alcohol, PEG-400 or propylene glycol, are regarded generally as safe and stable.

All the preparations are physically stable at +40° C.

Lipophilic cream—A water in oil (w/o) cream containing heavy paraffin oil was prepared using a silicone polymer emulsifier (cetyldimethicone copolyol and polyglyceryl-4-isostearate, Abil WE09). The advantage of this system is that a no heating is required during the process and the emulsion is very stable.

Three variants of the above formulation, with various consistencies (viscosity), were prepared.

Silicone polymer is widely used as emulsifier in cosmetics, however it is not registered in any Pharmacopea. Therefore, the emulsifier was replaced by sorbitan sesquioleate (Arlacel 83), a nonionic emulgator specific for emulsion w/o. The oily phase containing paraffin oil, and a synthetic wax: hydrogenated castor oil (castor wax) was used as a thickener.

All the preparations were found to be physically stable at +40° C.

Preparation of Prototypes on Laboratory Scale

According to the results of the preliminary formulations, each general formula (hydrophilic gel and lipophilic cream) was prepared as a placebo, 0.01% halofuginone and 0.03% halofuginone, on laboratory scale.

Hydrophilic Gel Prototype

The placebo was prepared in a 200 g-batch size. The procedure was performed at room temperature (RT). After dispersion of the Natrosol 250 HX in water, the solvents were added. The initial pH (7.4) was adjusted with a few drops of lactic acid 90% to the desired pH of about 4.0.

The drug products were similarly prepared (for example, 0.03% halofuginone, Table 3). The active ingredient, halofuginone-HBr, was accurately weighed elsewhere (Aminolab). The drug was dissolved in a mixture of solvents: water+propylene glycol+lactic acid (for pH adjustment), under stirring and gentle heating to 35-408/18/2004° C. The obtained solution was added to the finished product and homogenized by means of a homogenizer Silverson L4R.

The final product was packed in aluminum tubes (aluminum coated with epoxy resin), 3 g each:
Placebo gel-50 tubes.
Halofuginone gel 0.03% w/w-124 tubes.

Halofuginone gel 0.01% w/w was filled in 7 glass vials of 20 g each.

TABLE 3
Preparation of 0.03% halofuginone gel.

| | | | | Quantity for 500 g | | |
|---|---|---|---|---|---|---|
| | Raw Materials | | | | | Qty. for |
| No. | Name of Ingredient | Source | Lot | Calc. | Real | 100 g |
| 1 | Natrosol 250 HX Pharm. | Hercules | S-1972 | 10.00 | 10.0 | 2.0 |
| | Hydroxyethylcellulose | | | | | |
| | Ph. Eur. | | | | | |
| 2 | Water purified | Dr. Kadir | | 265.0 | 265.0 | 53.0 |
| 3 | Propylene glycol | Gadot | CO99113501 | 80.00 | 80.0 | 16.00 |
| | USP | | | | | |
| 4 | PEG 400 RENEX | UNIQEMA | 4253 | 100.0 | 100.0 | 20.0 |
| | Solution of the Active | | | | | |
| | Ingredient-HFBr | | | | | |
| 1 | Propylene glycol | Gadot | CO99113501 | 20.00 | 20.0 | 4.00 |
| | USP | | | | | |
| 2 | Water purified | Dr. Kadir | | 20.00 | 20.0 | 4.00 |
| 3 | Halofuginone-HBr | #5A303413 | EE028 | 0.150 | 150 mg | 0.03 |
| 4 | Lactic acid 90% | MERCK | K25707466 | 0.05 | 0.10 | 0.01 |
| | Ph. Eur. | | | | | |
| 5 | Water up to 100 | Dr. Kadir | | 4.80 | | 0.07 |
| | | | Total | 500.0 | 500.0 | 100.00 |

Lipophilic w/o Cream prototype

The placebo was prepared in a 500 g-batch size. The oily and the aqueous phases were heated separately to 75-80° C. The aqueous phase was then added slowly to the oily phase under a vigorous stirring. The viscosity of the cream increased with the addition of the aqueous phase.

The creams with the active drug were similarly prepared (for example, 0.03% halofuginone, Table 4).

The drug, (pre-weighed by Aminolab), was dissolved in a mixture of solvents: water+propylene glycol+lactic acid 90%. The pre-dissolved drug was added to the prepared cream after it was cooled to a temperature below 35° C. and homogenized by means of homogenizer Silverson L4R.

The final product was packed in aluminum tubes, 3 g each.
Placebo cream-Lot HF-30050; 70 tubes.
Halofuginone cream 0.03% w/w-Lot HF-Lot 60600; 107 tubes.

Halofuginone cream 0.01% w/w-HF-Lot 140600-1 was filled in 7 glass vials of 20 g each.

TABLE 4
Preparation of 0.03% halofuginone cream

| | | | | Quantity for 500 g | | |
|---|---|---|---|---|---|---|
| | | | | | | Obser. |
| | Raw Materials | | | | | Qty. for |
| No. | Name of Ingredient | Source | Lot | Calc. | Real | 100 g |
| | A. Hydrophobia Phase | | | | | |
| 1 | Sorbitan sesquioleate | UNIQEMA | FE591 | 25.00 | 25.01 | 5.00 |
| | Arlacel-83 | ICI-UK | | | | |
| 2 | Hydrogenated castor oil | HENKEL | 1648154 | 15.00 | 15.00 | 3.00 |
| | wax | Care | 3.08.98 | | | |
| | CUTINA HR PULVER | Spec. | | | | |
| | | Zifroni | | | | |
| 3 | Paraffin oil heavy | Esso | 08820154 | 125.0 | 125.1 | 25.00 |
| | BP; Ph. Eur. PRIMOL 352 | Paz | | | | |
| | | Chem. | | | | |
| 4 | Propyl paraben BP | Zifroni | PP 511299 | 0.50 | 0.50 | 0.10 |
| | B. Hydrophilic Phase | | | | | |
| 4 | Purified water | Dr. Kadir | | 300.0 | 300.0 | 60.00 |
| 5 | Magnesium sulfate | Merck | K26028882 | 2.50 | 2.5 | 0.50 |
| | heptahydrate BP | | | | | |
| 6 | Propylene glycol | Gadot | CO99113501 | 20.0 | 20.0 | 4.00 |
| | USP | | | | | |
| 7 | Methyl paraben BP | Zifroni | MP | 0.50 | 0.5 | 0.10 |
| | | | 071299 | | | |
| | C. Adjustment | | | | | |
| 8 | Water | Dr. Kadir | — | 5.60 | 5.6 | 1.12 |
| 9 | Propylene glycol | Gadot | CO99113501 | 5.00 | 5.0 | 1.00 |
| | USP | | | | | |
| 10 | Lactic acid 90% BP | Merck | K25707466 | 0.75 | 0.5 | 0.15 |
| 11 | Halofuginone HBr | #5A303413 | EE028 | 0.150 | 150 mg | 0.03 |
| | | TOTAL | | 500.0 | 500.0 | 100.00 |

Chemical Stability Study of the Cream and Gel Prototypes

The stability of the prototypes was measured under various conditions at a short time program. After two months the assays was stopped due to a decrease in halofuginone concentration under accelerated conditions (+40° C.). As shown in tables 5 and 6, halofuginone stabilized by lactic acid was more stable when formulated in the hydrophilic gel compared to the lipophilic cream: after 2 month under +40° C., only 5.5% of the halofuginone were degraded (compared to 12.5% in the cream formulation).

TABLE 5
Stability of 0.03% halofuginone in gel
Batch No. HF-70600 (pH 3.5-4.5)

| | Storage Conditions | | |
|---|---|---|---|
| | ASSAY % | | |
| Interval Of Time - Months | +4° C. | +25° C. | +40° C. |
| Zero Time | 97.80 | 97.80 | 97.80 |
| 1 month | 98.10 | 99.10 | 91.50 |
| 2 months | _— | 99.20 | 94.50 |

TABLE 6
Stability of 0.03% halofuginone in
cream Batch No. HF-60600(PH 4)

| | Storage Conditions | | |
|---|---|---|---|
| | ASSAY % | | |
| Interval Of Time - Months | +4° C. | +25° C. | +40° C. |
| Zero Time | 98.00 | 98.00 | 98.00 |
| 1 month | 98.30 | 93.90 | 89.70 |
| 2 months | — | 94.80 | 87.50 |

Oral Administration
Tablets

In the formulation of tablets dosage form for oral administration the active ingredient is applied in its dried form. Surprisingly, it is now shown that even in a solid-state formulation halofuginone is more stable under acidic conditions.

Five batches of tablets as detailed in Table 7 herein below were placed under accelerated stability storage conditions (40° C. at 75% RH and at 55° C.). Halofuginone stability was measured after 10 days. The amount of the cis isomer of halofuginone is a criterion of loss of activity, due to isomerization of the pharmaceutically active trans-halofuginone; therefore, a higher amount of the cis isomer during stability studies or drug storage indicates non-stabilizing formulations.

TABLE 7
Stability of halofuginone in tablets dosage form

| | Amount | Amount | Amount in | Amount | Amount |
|---|---|---|---|---|---|
| | in tablet (mg) | in tablet (mg) | tablet (mg) | in tablet (mg) | in tablet (mg) |
| | High talc | Low talc | Low talc | Low talc | Low talc |
| | No acid | No acid | 0.25% Acid | 1% Acid | 2% Acid |
| Batch # | 8797 | 8796 | 8822 | 8824 | 8823 |
| Halofuginone | 0.5 | 0.5 | 0.5 | 0.5 | 0.5 |
| Citric Acid | — | — | 0.5 | 2 | 4 |
| Mg Stearate NF | 4 | 2 | 2 | 2 | 2 |
| Talc USP | 40 | 4 | 4 | 4 | 4 |
| Microcrystaline | 20 | 89.3 | 89.3 | 89.3 | 89.3 |
| Cellulose NF | | | | | |
| Crospovidine | 4 | 4 | 4 | 4 | 4 |
| NF | | | | | |
| Colloidal | 0.3 | 0.2 | 0.2 | 0.2 | 0.2 |
| Silicon Dioxide | | | | | |
| NF | | | | | |
| Lactose | 131.2 | 100 | 99.5 | 98 | 96 |
| Monohydrate | | | | | |
| NF | | | | | |
| Total | 200 | 200 | 200 | 200 | 200 |

Stability results

| | | | | | |
|---|---|---|---|---|---|
| Isomer cis-55° C. | 3.1 | 1.8 | 1.26 | 0.45 | 0.3 |
| Isomer cis-40° C.- | 1.3 | 0.7 | NP | 0.15 | NP |
| 75% RH | | | | | |

It is seen from these results that even in solid-state formulation, the stability of halofuginone is depended on acidic medium in the formula, as represented by formation of cis isomer under even slight basic conditions.

The worst results observed in tablets with high amount of talc without addition of acid, the basic talc has accelerating effect on the drug isomerization and cis isomer formation. Addition of citric acid decreased the formation of cis isomer and therefore stabilized the drug in this formula.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. A pharmaceutical composition comprising as an active ingredient a quinazolinone derivative having the general formula (I): 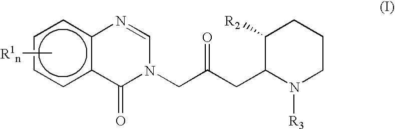
wherein: n=1-2
R₁ which at each occurrence is independently selected from the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof, in a pharmaceutical acceptable carrier or diluent, further comprising an acid compound, providing a pH below 7.0.

2. The pharmaceutical composition according to claim 1 wherein the active ingredient is halofuginone or pharmaceutically acceptable salt of halofuginone.

3. The pharmaceutical composition according to anyone of claims 1-2 for topical, parenteral or oral administration.

4. The pharmaceutical composition according to anyone of claims 1-3 wherein the pH of the composition is below 6.0.

5. The pharmaceutical composition according to claim 4 wherein the pH of the composition is below 5.5.

6. The pharmaceutical composition according to anyone of claims 1-3, wherein the concentration of the active compound is in the range of 0.0001-30%.

7. The pharmaceutical composition according to anyone of claims 1-3, wherein the concentration of the active compound is in the range of 0.001-10%.

8. The pharmaceutical composition according to claim 3 wherein the topical form is selected from the group consisting of cream, ointment, lotion, gel, suspension, aqueous or cosolvent solution, salve and liposomes.

9. The pharmaceutical composition according to claim 8 wherein the concentration of the active compound is in the range of 0.001-2%.

10. The pharmaceutical composition according to claim 8 wherein the composition is a cream comprising in addition to the active compound, a hydrophobic component, a hydrophilic component and at least one emulsifying agent.

11. The pharmaceutical composition according to claim 10 wherein the hydrophobic component is selected from the group consisting of mineral oil, yellow soft paraffin, white soft paraffin, paraffin (hard paraffin), paraffin oil heavy, hydrous wool fat (hydrous lanolin), wool fat (lanolin), wool alcohol (lanolin alcohol), petrolatum and lanolin alcohols, beeswax, cetyl alcohol, almond oil, arachis oil, castor oil, hydrogenated castor oil, cottonseed oil, ethyl oleate, olive oil, sesame oil, and mixtures thereof.

12. The pharmaceutical composition according to claim 10 wherein the hydrophilic component is selected from water, propylene glycol, a pharmaceutically acceptable buffer and mixtures thereof.

13. The pharmaceutical composition of claim 12 wherein the hydrophilic component is lactate buffer.

14. The pharmaceutical composition according to claim 10 wherein the emulsifying agent is a complex emulgator which comprises a combination of a hydrophilic and a hydrophobic emulsifying agent.

15. The pharmaceutical composition according to claim 14 wherein the hydrophilic emulsifying agent is selected from the group consisting of polyoxyethylene sorbitan monolaurate, polyoxyethylene sorbitan monopalmitate, polyoxyethylene sorbitan monostearate, polyoxyethylene sorbitan monooleate, polyoxyethylene lauryl ether, polyoxyethylene castor oil, sodium lauryl sulfate, cetrimide, cetomacrogol and mixtures thereof.

16. The pharmaceutical composition according to claim 14 wherein the hydrophobic emulsifying agent is selected from the group consisting of sorbitan trioleate, sorbitan sesquioleate, sorbitan tristearate, sorbitan monooleate, propylene glycol monostearate, sorbitan sequioleate, glycerol monostearate, propylene glycol monolaurate, sorbitan monostearate, sorbitan monopalmitate, sorbitan monolaurate, cetostearyl alcohol, cetyl alcohol, oleic acid, stearic acid and mixture thereof.

17. The pharmaceutical composition according to claim 8 wherein the composition is an aqueous suspension comprising at least one agent selected from suspending agents, thickeners, wetting agents and flocculating agents.

18. The pharmaceutical composition according to claim 17, wherein the suspending agents or thickeners are present in an amount from about 0.1% to about 15% (w/w).

19. The pharmaceutical composition according to claim 17, wherein the suspending agents or thickeners are selected from the group consisting of cellulose derivatives, methylcellulose, hydroxyethylcellulose, hydroxypropyl cellulose, alginic acid and its derivatives, xanthan gum, guar gum, gum arabic, tragacanth, gelatin, acacia, bentonite, starch, microcrystalline cellulose, povidone and mixtures thereof.

20. The pharmaceutical composition according to claim 8 wherein the composition is a gel comprising an aqueous medium and at least one gelling agent.

21. The pharmaceutical composition according to claim 20 wherein the gelling agent is present in an amount from about 1% to about 25% (w/w).

22. The pharmaceutical composition according to claim 20 wherein the gelling agent is selected from the group consisting of hydrophilic polymers, natural and synthetic gums, crosslinked proteins and mixture thereof.

23. The pharmaceutical composition according to claim 22 wherein the polymers are selected from the group consisting of hydroxyethylcellulose, hydroxyethyl methylcellulose, methyl cellulose, hydroxypropylcellulose, hydroxypropyl methylcellulose, carboxymethyl cellulose, derivatives of amylose, dextran, chitosan, pullulan, and other polysaccharides; crosslinked proteins, albumin, gelatin and collagen; acrylic based polymer gels, hydroxyethyl methacrylate based gel polymers, polyurethane based gels and mixtures thereof.

24. The pharmaceutical composition according to claim 22 wherein the gums are selected from the group consisting of acacia, agar, carageenan, dextrin, gelatin, guar gum, hyaluronic acid, tragacanth gum, xanthan gum and mixture thereof.

25. The pharmaceutical composition according to claim 8 wherein the composition is a solution.

26. The pharmaceutical composition according to claim 25 wherein the solvent is selected from the group consisting of water, buffered solutions, organic solvents, ethyl alcohol, isopropyl alcohol, propylene glycol, polyethylene glycol, glycerin, glycoforol, ethyl lactate, methyl lactate, N-methylpyrrolidone, ethoxylated tocopherol and mixtures thereof.

27. The pharmaceutical composition according to any one of claims 17-26 wherein the acid compound is selected from the group consisting of aliphatic, aromatic, acetic, glycolic, lactic, malic, maleic, citric, ascorbic or benzoic acid.

28. The pharmaceutical composition according to claim 3 wherein the composition is a parenteral formulation suitable for intravenous infusion; intravenous, intraperitoneal, intramuscular, subcutaneous injections or depots, or for administration laparascopially and intravesicularly.

29. The pharmaceutical composition according to claim 28 wherein the pharmaceutical composition is selected from the group consisting of sterile solutions ready for injection, sterile suspensions ready for injection, sterile dry soluble lyophilized powders ready for reconstitution by combination with a vehicle just prior to use, sterile emulsions, microemulsions, dispersions, liposomal dosage forms and lipid complexes.

30. The pharmaceutical composition according to claim 29 wherein the solutions are selected from the group consisting of sterile water for injection, sodium chloride injection, dextrose and lactated Ringers injection.

31. The pharmaceutical composition according to claims 28-30 wherein the acid compound is selected from the group consisting of glycolic, lactic, malic, maleic, citric, ascorbic, benzoic.

32. The pharmaceutical composition according to claim 3 wherein the composition is an oral formulation selected from solid or aqueous forms.

33. The pharmaceutical composition according to claim 32 wherein the solid formulation is selected from the group consisting of tablets, capsules, sachets, powders, granules and lozenges.

34. The pharmaceutical composition according to claim 33 optionally further comprising lubricating agents, wetting agents, emulsifying and suspending agents, preserving agents, sweetening agents, polyols, buffers and inert fillers.

35. The pharmaceutical composition according to claim 34 comprising at least one filler selected from starches, gum arabic, calcium silicate, microcrystalline cellulose, PVP, cellulose and methyl cellulose.

36. The pharmaceutical composition according to claim 34 wherein the preserving agent is selected from the group consisting of methyl- and propyl hydroxybenzoate.

37. The pharmaceutical composition according to claim 34 wherein the polyols are selected from the group consisting of mannitol, sorbitol, xylitol, sucrose, maltose, glucose, lactose and dextrose.

38. The pharmaceutical composition according to claims 33-37 wherein the acid compound is selected from the group consisting of citric, stearic, ascorbic, lactic, malic and maleic acid.

39. The pharmaceutical composition according to claim 33 wherein the formulation is selected from the group consisting of sterile solutions, sterile suspensions, sterile dry soluble lyophilized powders ready for reconstitution by combination with a vehicle just prior to use, sterile emulsions, microemulsions, dispersions, liposomal dosage forms and lipid complexes.

40. A method of improving stability of pharmaceutical composition comprising as an active ingredient a component of the general formula (I) 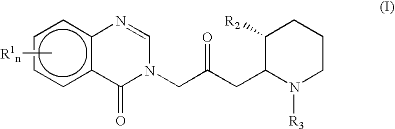
wherein: n=1-2
R₁ which at each occurrence is independently selected from the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkoxy-carbonyl, and pharmaceutically acceptable salts thereof, in a pharmaceutical acceptable carrier or diluent, comprising adding an acid component providing a pH below 7.0.

41. The method of claim 40 wherein the compound of general formula I is halofuginone, or a pharmaceutically acceptable salt thereof.

42. The method of claim 40 or 41 wherein the pH is below 6.0.

43. The method of claim 42 wherein the pH is below 5.5.
